(19) United States
(12) Patent Application Publication
(10) Pub. No.: US 2010/0028310 A1
(43) Pub. Date: Feb. 4, 2010

(54) Composition for Transplantation Comprising Adipose Stem Cells or Adipocytes

(76) Inventors: Byung Rok Do, Seoul (KR); Ji Hyang Kim, Seoul (KR); Sung Goo Kang, Busan (KR); Chul Geun Kim, Seoul (KR); Hyuck Chan Kwon, Seoul (KR); Byeong Kyu Kim, Seoul (KR); Ai-Young Lee, Goyang-si (KR); Jea Kyung Sung, Changwon (KR)

Correspondence Address: FROMMER LAWRENCE & HAUG, 745 FIFTH AVENUE- 10TH FL., NEW YORK, NY, 10151, US

(21) Appl. No.: 12/304,813

(22) PCT Filed: Jun. 5, 2007
(86) PCT No.: PCT/KR2007/002729

§ 371 Date: Jul. 6, 2009

(30) Foreign Application Priority Data
Jun. 14, 2006 (KR) 10-2006-0053247
May 4, 2007 (KR) 10-2007-0043363

(51) Int. Cl. A61K 35/12; A61P 17/00

(52) U.S. Cl. 424/93.7

(57) ABSTRACT

The present invention relates to a composition for transplantation in a physiologically compatible buffer solution comprising, (a) a transplant-cell selected among an adipose stem cell, an adipocyte, adipose tissues (fat tissue) and a mixture thereof; and (b) a semi-solid substance derived from a living body or a biodegradable substance selected among a hyaluronic acid, collagen, elastin, thrombin, chondroitin sulfate, albumin and a mixture thereof.

### TECHNICAL FIELD

The present invention relates to a stable composition for transplantation comprising adipose stem cells, adipocytes, separated adipose tissues (aspirated fat tissues) or a mixture thereof.

### RELATED PRIOR ART

Transplantation of adipose tissue in human has been commonly performed using subcutaneous fat separated, for example, from the abdomen or the legs for the purpose of cosmetic surgeries of a face or breasts or plastic surgeries for correcting body shapes or male genital organs or removing wounds and/or scars. Generally, subcutaneous fat transplantation is performed by separating subcutaneous fat using a blunt-ended needle such as a tulip needle. Subcutaneous fat is separated by centrifugation into blood and adipose tissues, and the adipose tissues are transplanted into a targeted body region with a needle.

The conventional transplantation using subcutaneous fat have drawbacks that many adipocytes may be damaged while collecting the subcutaneous fat, it is difficult to precisely transplant a desired amount to the target area due to the presence of remaining cell masses in various size; and the cell mass may be recognized in areas with thin-skin having a relatively small amount of subcutaneous fat (e.g., areas below the eyes). Further, when only the mass of subcutaneous fat is transplanted, adipocytes present within adipose tissues mass may not be able to obtain necessary nutrients or discharge body wastes, thus readily being led to death.

Therefore, when adipose tissues mass is separated into adipocytes and/or adipose stem cells for transplantation, it may be able to overcome the problem due to a direct transplantation of subcutaneous fat to a patient. Further, stem cells derived from fat, e.g., adipose stem cells, may be differentiated into adipocytes, which, being small with a size of less than 10 μm initially, can grow into cells with a size of more than 100 μm. The volumes may increase about 1,000 times theoretically, and may be used with preference.

However, in case infusion for transplantation is prepared by dispersing adipocytes and/or adipose stem cells in a normal (physiological) saline solution such as Ringer's solution, the aforementioned cells may easily precipitate, thus making it difficult to inject a uniform number of adipose stem cells into targeted regions. Further, the differentiation of adipose stem cells into matured adipose cells at transplanted regions may cause a serious imbalance.

Moreover, transplantations at hips or breasts, which require a relatively higher amount of fat, may be performed by extracting stem cells using some of extracted adipose tissues and mixing them with the extracted adipose tissues to increase the concentration of stem cells. However, this method also has a disadvantage that stem cells and pre-existing adipose tissues may not be uniformly distributed in the mixture.

Thus, the present inventors made intensive efforts to develop a composition for transplantation, where adipocytes, adipose stem cells and/or adipose tissues are stably dispersed. As a result, the present invention was completed by finding that adipocytes and/or adipose stem cells can be stably dispersed in a medium such as a buffer solution, when it contains a highly viscous semi-solid substance derived from a living body (organism) or a biodegradable substance such as hyaluronic acid, albumin, collagen, elastin and chondroitin sulfate with high viscosity.

Therefore, the present invention relates to a stable composition for transplantation comprising adipocytes, adipose stem cells and/or adipose tissues.

### DETAILED DESCRIPTION

According to an aspect of the present invention, there is provided a composition for transplantation comprising (a) a transplant-cell selected from the group consisting of adipose stem cells, adipocytes and a mixture thereof; and (b) a semi-solid substance derived from a living body or a biodegradable substance selected from the group consisting of hyaluronic acid, collagen, thrombin, elastin, chondroitin sulfate, albumin and a mixture thereof, in a physiologically compatible buffer solution.

Hereunder is provided a detailed description of the present invention.

In the present invention, adipocytes, adipose stem cells and adipose tissues may be prepared according to the conventional method. For example, as described in PCT publication Nos. WO2000/53795 and WO2005/042730, adipocytes and adipose stem cells may be separated from adipose tissues by means of liposuction, precipitation, treatment with an enzyme such as collagenase, removal of a supernatant such as red blood cells via centrifugation.

In the present invention, physiologically compatible buffer solution comprises buffer solution, which is usually used for a transplantation infusion such as medical or blood injection infusion, for example, a physiological saline solution having the same osmotic pressure as that of body fluids.

A composition herein may comprise, as a transplant cell, each of adipose stem cells, adipocytes, adipose tissues; a mixture of adipocytes and adipose stem cells; a mixture of adipocytes and adipose tissues; a mixture of adipose stem cells and adipose tissues; or a mixture of adipose stem cells adipocytes and adipose tissues. In particular, when both adipose stem cells and adipocytes are contained, the composition may serve the purpose of transplantation and may also increase the volume of the transplanted part due to adipocytes. Further, adipose stem cells may be differentiated and grown to promote the volumetric growth of the transplanted part. Further, in case of plastic surgeries in breasts and hips, where a relatively large amount of adipocytes is conventionally transplanted deep in skin, it is preferable to transplant all of extracted stem cells, adipocytes and adipose tissues at the same time.

When preparing adipose stem cells, adipocytes and/or adipose tissues, it is preferable to minimize the amount of adipocytes, which are damaged during the collection of adipose tissues from subcutaneous fat. As used herein, “minimize the damaged adipocytes” means to treat the substance so that oil or cell debris derived from adipocytes may remain in the amount of less than 30%, less than 20%, less than 10%, less than 5%, less than 3%, preferably less than 1%.

The amounts of adipose stem cells, adipocytes, adipose tissues and a mixture thereof in a composition herein may be determined considering the physical conditions, sex, and age of a patient or the object of transplantation. For example, for the purpose of cosmetic surgery of a face, adipocytes may be transplanted in the amount of about 1-100 mL, preferably about 3-50 mL. Adipose stem cells may be used in the amount of 1×10³-10⁸ cells. Further, when both adipocytes and adipose stem cells are used, they may be mixed without limitation within the aforementioned range for use. For example, when both adipocytes and adipose stem cells are used, about 50 mL of adipocytes and about 1×10⁵ cells of adipose stem cells may be used. One of the advantages of a composition for transplantation herein is that the mixing ratio between adipocytes and adipose stem cells can be appropriately adjusted to be suitable for transplantation.

In the present invention, the semi-solid substance derived from a living body or a biodegradable substance is a stabilizer for stably dispersing the transplant cells. Because this is derived from a living body, it is biodegradable and shows no side effect when transplanted into body substance. As used herein, the “biodegradable” refers to being slowly degraded and absorbed into a body, and there is no limitation in the degradation rate. Examples of the semi-solid substance derived from a living body or a biodegradable substance include hyaluronic acid, collagen, thrombin, elastin, chondroitin sulfate, albumin and a mixture thereof, and hyaluronic acid or collagen is preferred. Hyaluronic acid, collagen, thrombin, elastin, chondroitin sulfate or albumin is derived from a living body, and naturally degrades in a body. A composition for transplantation herein may be endowed with a semi-floating property by maintaining high viscosity when contained in hydrophilic medium. However, even synthetic compounds may be biodegradable substance as long as it is biodegradable and satisfies the viscosity-conferring property. Because this synthetic compound may also serve the purpose of the present invention, the biodegradable substance is not limited to a substance derived from a living body in the present invention. Therefore, when these are formulated in combination with adipocytes, adipose stem cells and/or adipose tissues as in a composition herein, it is preferred to uniformly disperse adipocytes, adipose stem cells and/or adipose tissues without floating or precipitating in a medium.

The semi-solid substance derived from a living body or a biodegradable substance contained in a composition herein is preferred to have a predetermined concentration or a weight average molecular weight to provide such a viscosity that adipocytes, adipose stem cells and/or adipose tissues may be stably dispersed.

For example, when a composition herein contains only adipose stem cells as a transplant cell, the semi-solid substance derived from a living body or a biodegradable substance may be contained in an amount of about 0.001-20 wt %, preferably about 0.05-5 wt %, more preferably about 0.1-2 wt % relative to the total weight of the composition.

Further, in case a composition herein contains adipocytes and/or adipose tissues; or all of adipose stem cells and adipocytes and/or adipose tissues as a transplant cell, the semi-solid substance derived from a living body or a biodegradable substance may be contained in an amount of about 0.0001-10 wt %, preferably about 0.001-1 wt %, more preferably about 0.002-0.4 wt %, most preferably about 0.02-0.2 wt % relative to the total weight of the composition.

A composition herein may comprise the semi-solid substance derived from a living body, i.e., hyaluronic acid, collagen, thrombin, elastin, chondroitin sulfate, albumin and synthetic biodegradable substance or an equivalent substance thereof. Of these, hyaluronic acid or collagen may be preferred.

Hyaluronic acid is a biodegradable polymer derived from a living body approved to be injected into a human body by the Korea Food and Drug Administration (KFDA) and US Food and Drug Administration (USFDA). In the present invention, hyaluronic acid can be selected from the group consisting of a purified hyaluronic acid separated from body liquids or tissues of a mammal including human, a hyaluronic acid prepared by culturing recombinant microorganisms or cells, and a chemically synthesized hyaluronic acid. Hyaluronic acid may have a weight average molecular weight of about 200,000-400,000, which may be modified depending on the content, i.e., concentration, of hyaluronic acid in a composition herein.

Collagen is a scleroprotein contained in large amount in bones, cartilage, tendon and skin of mammals including human, which is transformed into gelatin when melted. In the present invention, collagen may be selected among collagen obtained from a mammal including human and collagen prepared by cultivating recombinant microorganisms or cells. Collagen may have a molecular weight of 100-200 kDa, preferably about 130 kDa, which may be modified depending on its content, i.e., concentration of collagen in a composition herein.

Albumin is present in large amount in blood of mammals including human. In the present invention, albumin may be selected among albumin obtained from body fluids of a mammal including human and albumin prepared by cultivating recombinant microorganisms or cells. In the present invention, albumin is preferred to have a predetermined concentration and a molecular weight to provide such a viscosity that adipocytes and/or adipose stem cells may be stably dispersed. This concentration is higher than that of the semi-solid substance derived from a living body or a biodegradable substance. That is, for example, albumin may be used in an amount of about 0.05-50 wt %, preferably 1-30 wt %, more preferably about 20 wt % relative to the total weight of the composition. Further, albumin may has a molecular weight of about 60-90 kDa, which may be appropriately selected considering the content, i.e., concentration of albumin in a composition. Further, commercially available albumin, for example albumin human fraction V (Promega, USA) may be used in the present invention.

A composition herein may optionally comprise pharmaceutically acceptable additives. Examples of the additives include without limitation an isotonic agent such as dextran and a soothing agent such as benzyl alcohol.

A composition herein comprises adipose stem cells, adipocytes and/or adipose tissues that are stably dispersed without floating or precipitating in a physiologically compatible buffer solution. Therefore, a composition for transplantation of the present invention enables the minute control of the content of transplant cell, and may be injected into various transplantation areas in an exact amount, thereby remarkably decreasing side effects of a composition for transplantation herein.

Further, the uniform introduction of transplant cells prevents non-uniform transplantation of adipose stem cells, adipocytes and/or adipose tissues. Through the spaces of the transplanted cells, stem cells and adipocytes may easily receive nutritive elements from outer body fluids or discharge body wastes. Furthermore, the introduced adipocytes and stem cells may extend through the pre-existing adipose tissues and connective tissues and be assisted by the blood vessels, thereby increasing the survival rate of cells and lowering the volumetric decrease after the transplantation.

### BRIEF DESCRIPTION OF DRAWINGS

FIG. 1 shows the separation of two layers generated when the collected adipose tissues were added with a physiological buffer solution, wherein the upper one is adipose tissues layer, and the lower one is a layer comprising body fluids including blood, substances that may be added during tissue separation, and a physiologically suitable buffer solution (the red-colored layer is a mixture of body fluids and a buffer solution).

FIG. 2 shows the separation of layers after the removal of body fluids including blood and substances that may be added during tissue separation, wherein the upper layer is adipose tissues layer and the lower one is a layer having a physiologically suitable buffer solution.

FIG. 3 shows the separation of layers after the adipose tissues were treated with collagenase, wherein the upper layer is an oil layer derived from damaged adipocytes; the intermediate layer is an adipocyte layer; and the lower layer is an adipose stem cell layer.

FIG. 4 shows the separation of a composition for transplantation according to one embodiment of the present invention, which comprises hyaluronic acid and a physiological saline solution in addition to adipocytes and adipose stem cells obtained from each layer shown in FIG. 3.

FIG. 5a is a graph showing the weight percentages of adipose stem cells contained in each of the four equally-divided volumes, wherein the composition for transplantation was divided into the uppermost layer, the upper intermediate layer, the lower intermediate layer and the lowermost layer immediately after placing a composition for transplantation containing adipose stem cells, a physiological saline solution, and with a biodegradable stabilizer (with hyaluronic acid, left) and a composition without a biodegradable stabilizer for transplantation (without hyaluronic acid, right) into a test tube (0 hour).

FIG. 5b is a graph showing the distribution of adipose stem cells contained in the aforementioned specimen after 24-hour storage at room temperature, wherein the composition for transplantation was divided into the uppermost layer, the upper intermediate layer, the lower intermediate layer and the lowermost layer immediately after the preparation of adipocytes containing-composition for transplantation without a semi-solid substance derived from a living body and after 24-hour storage.

FIG. 6 is a graph showing the weight percentages of adipocytes contained in each of the four equally-divided volumes.

FIG. 7 shows the microphotographs, taken immediately after the preparation and after 24-hour storage, of the respective uppermost layer and the respective lowermost layer of a composition for transplantation containing adipocytes shown in FIG. 6.

FIG. 8 shows the microphotographs, taken immediately after the preparation and after 24-hour storage, of the respective uppermost layer and the respective lowermost layer of adipose tissues-containing composition for transplantation containing no semi-solid substance derived from a living body.

### EXAMPLES

The present invention is described more specifically by the following Examples. Examples herein are meant only to illustrate the present invention, but they should not be construed as limiting the scope of the claimed invention.

### Example 1

### Collection of Adipocytes and Adipose Stem Cells

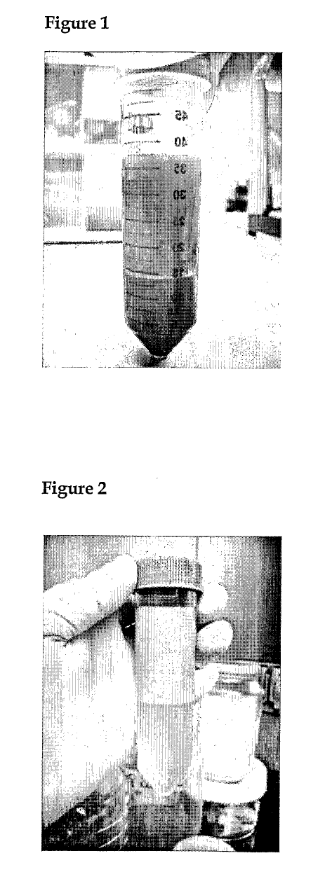

Adipose tissues, which had been removed from a patient through liposuction and discarded, were collected from a patient after obtaining his/her approval. The collected adipose tissues were divided into 10-200 g aliquots, and each aliquot was moved into a processing bag. The adipose tissues were added with a physiological buffer solution, and treated with a fresh physiological buffer solution three times to remove the body fluids and blood, thereby obtaining adipose tissues without body or blood. FIG. 1 shows the separation of the collected adipose tissues added with a physiological buffer solution. FIG. 2 shows the separation of the collected adipose tissues after the triple treatment of a physiological buffer solution. As shown in FIG. 1, adipose tissues float, and a substance derived from blood such as globules precipitated down in the buffer solution. Further, FIG. 2 shows that the separated adipose tissues contained non-uniform adipocytes, and are thus hard to be passed through an injection needle.

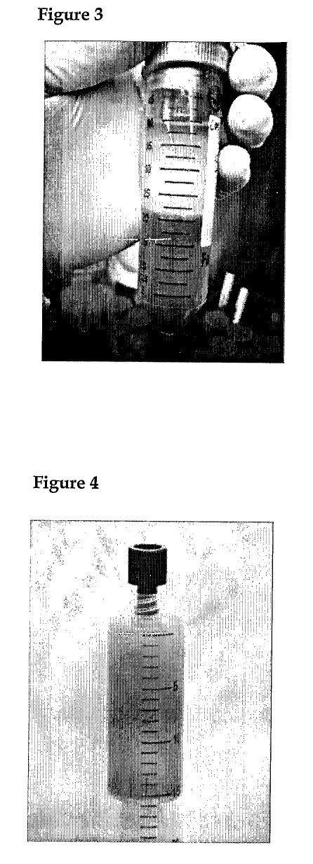

The adipose tissues containing neither blood fluids nor blood were collected, and added with collagenase to a final concentration of 0.05-0.2 wt %, and stirred at 37° C. for 1 hour. The adipocyte layer and the adipose stem cell layer were separated, and each layer was added with a physiological saline solution to remove collagenase. The adipose tissues were treated with collagenase, and divided into an oil layer (upper portion), an adipocyte layer (middle portion) and an adipose stem cell layer (lower portion). The result is shown in FIG. 3.

Thus prepared adipocyte layer was centrifuged (50×g) to remove damaged adipocytes and disrupted fat cells derived oil generated during the collection and the enzyme treatment processes. Further, obtained adipose stem cell layer was also centrifuged (50×g) to remove the damaged adipocytes and disrupted fat cells derived oil generated during the collection and the enzyme treatment processes.

### Example 2

### Preparation of Composition for Transplantation Containing Adipose Stem Cells

A composition for transplantation containing adipose stem cells was prepared by adding 2.5 mL of a physiological buffer solution containing adipose stem cells (1×10⁷) prepared in Example 1 in 2.5 mL of 2 wt % hyaluronic acid for medical injection into a body with a weight average molecular weight of 300,000 and stirring the mixture for 10 minutes.

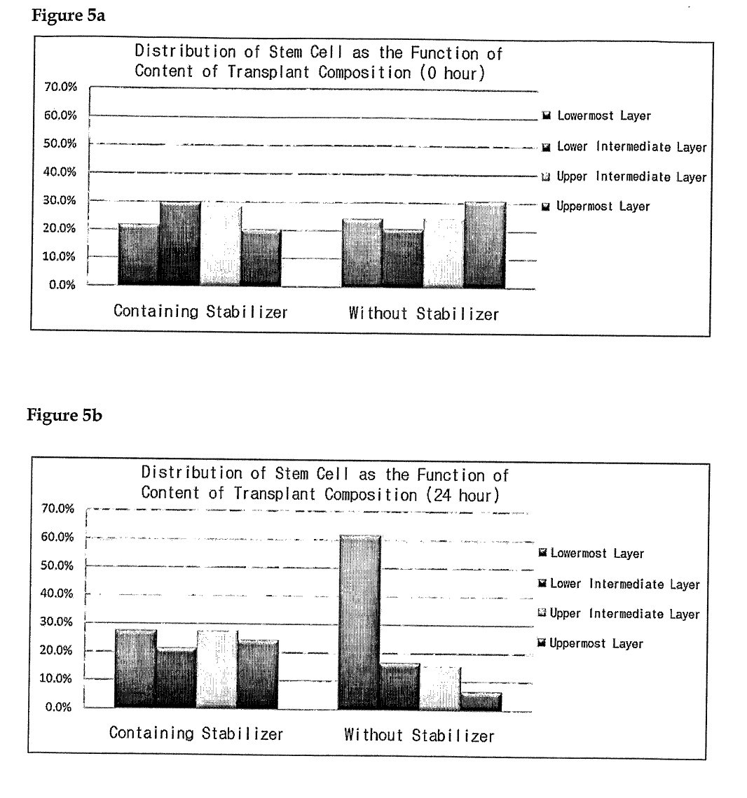

Thus prepared composition for transplantation was placed in a test tube (height of 10 cm and diameter of 0.4 cm), and the composition for transplantation was immediately (0 hour) divided equally into an uppermost layer, an upper intermediate layer, a lower intermediate layer and a lowermost layer. The weight of adipose stem cells contained in the same volume was weighed, and the distribution of the weight was provided in FIG. 5a (left side). After the specimen was stored at room temperature for 24 hours, the distribution of adipose stem cells was measured and is shown in FIG. 5b (left side). Meanwhile, the group of semi-solid substance derived from a living body, i.e., containing no stabilizer, which is presented in the right side of FIGS. 5a and 5b, is a composition for transplantation prepared by using 2.5 mL of a physiological buffer solution 2.5 mL in Example 2 instead of the hyaluronic acid for medical purpose. As compared to the stabilizer-containing group, this group was confirmed to have uniform distribution without showing any precipitation of adipose stem cells even at 24 hours from the preparation.

### Example 3

### Preparation of Composition for Transplantation Containing Adipocytes

A composition for transplantation containing adipocytes by adding 15 mL of adipocytes prepared in Example 1 in 0.5 mL of 2 wt % hyaluronic acid having a weight average molecular weight of 300,000 for medical purpose of body injections, and mixing the mixture for 10 minutes.

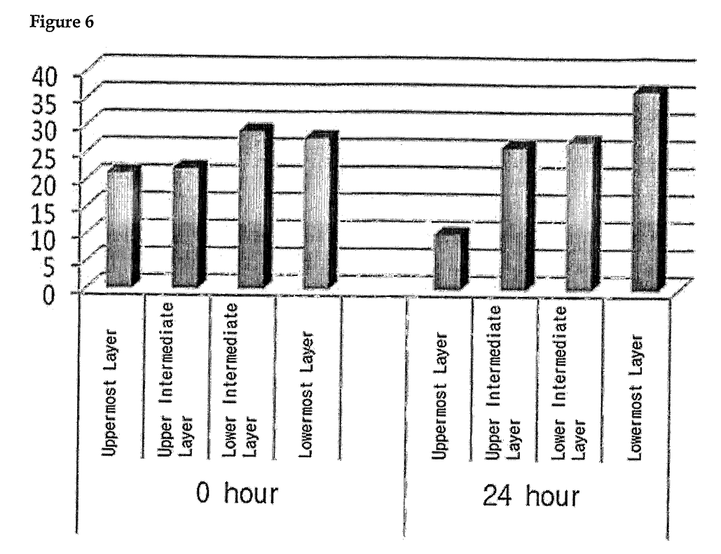

A composition for transplantation containing no semi-floating substance derived from a living body herein, which was prepared by adding 0.5 mL of a physiological buffer solution instead of hyaluronic acid of the present Example presented in FIG. 6, was confirmed to be uniformly present in all the layers in the amount of 20-30 wt % immediately after the preparation of the composition for transplantation. Twenty four hours after the preparation, adipocytes were confirmed to be present in the amount of less than 10 wt %, and more than 35 wt % in the uppermost layer and the lowermost layer, respectively.

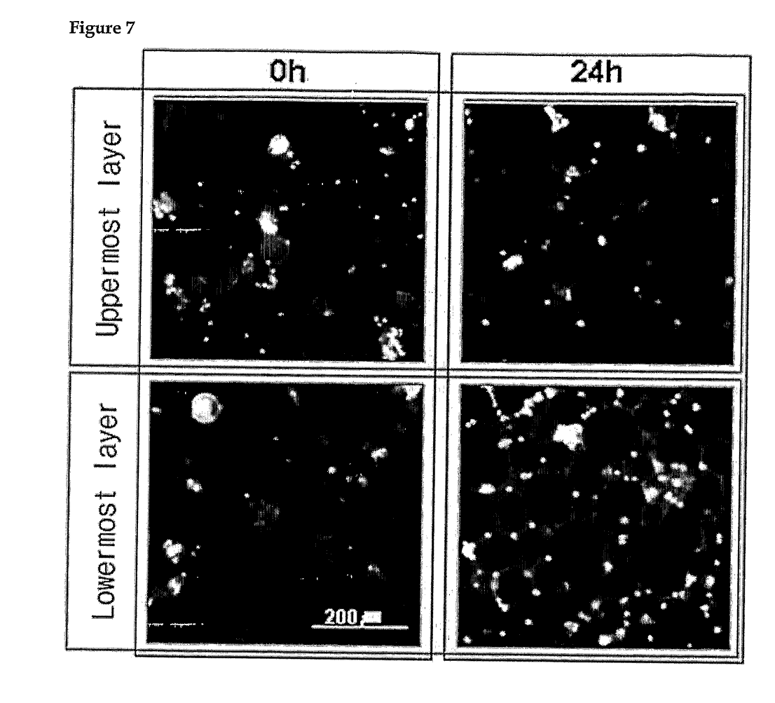

Further, as shown in FIG. 6, microphotographs of the uppermost layer and the lowermost layer were taken immediately after the preparation of the composition for transplantation and after 24-hour storage. The results presented in FIG. 7 showed that adipocytes were uniformly present in the uppermost layer and the lowermost layer, while they precipitated down to the lowermost layer after 24-hour of storage.

### Example 4

### Preparation of Composition for Transplantation Containing Adipocytes and Adipose Stem Cells

A composition for transplantation containing adipocytes and adipose stem cells was prepared by adding 0.5 mL of a physiological buffer solution containing adipose stem cells (1×10⁷) and 15 mL of adipocytes prepared in Example 1 in 0.5 mL of 2% hyaluronic acid having a weight average molecular weight of 300,000 for medical purpose of body injection, and stirring the mixture for 5 minutes.

FIG. 4 shows the separation of the composition for transplantation of the present Example containing hyaluronic acid and physiological saline solution in addition to adipocytes and adipose stem cells prepared in Example 1.

Thus prepared composition for transplantation was confirmed to be uniformly present throughout from the uppermost layer to the lowermost layer both immediately after the preparation and after 24-hour storage without causing separation between adipocyte layer and adipose stem cell layer.

### Example 5

### Preparation of Composition for Transplantation Containing Adipose Stem Cells

Collagen having a molecular weight of 130 kDa was dissolved in a physiological saline solution to provide a 2 wt % solution. This collagen solution (1 mL) was added with 1 mL of the physiological buffer solution containing adipose stem cells (1×10⁵) prepared in Example 1, followed by stirring for 10 minutes, thereby providing a composition for transplantation containing adipose stem cells.

### Example 6

### Preparation of Composition for Transplantation Containing Adipocytes and Adipose Stem Cells

A composition for transplantation containing adipocytes and adipose stem cells by adding 15 mL of the adipocytes prepared in Example 1 in the composition for transplantation prepared in Example 5, and stirring the mixture for 5 minutes.

### Example 7

### Preparation of Composition for Transplantation Containing Adipose Tissues and Adipose Stem Cells

A composition for transplantation was prepared by adding 0.5 mL of a physiological buffer solution containing adipose stem cells (1×10⁷) prepared in Example 1 and 15 mL of washed adipose tissues separated from a patient, which was obtained without collagenase treatment in 5 mL of 2 wt % hyaluronic acid having a weight average molecular weight of 300,000 for medical purpose of body injection, and stirring the mixture for 5 minutes.

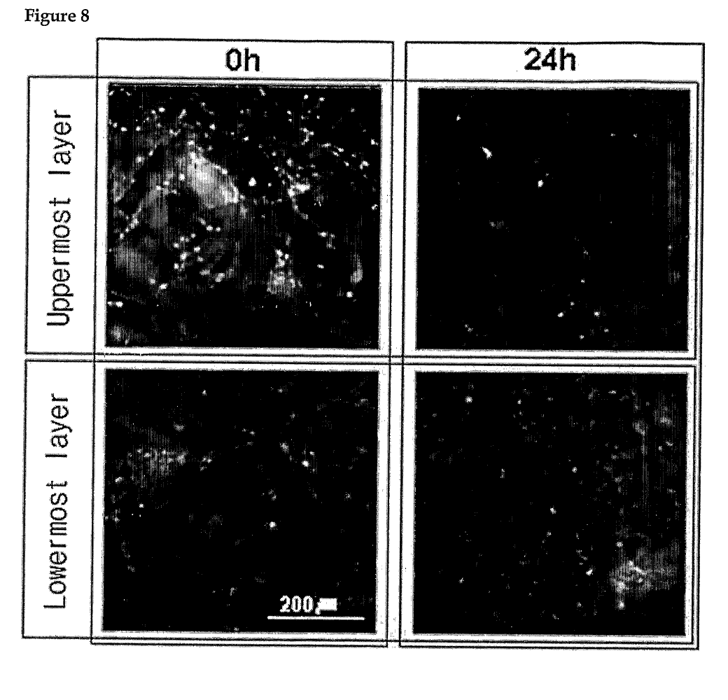

Meanwhile, microphotographs of the uppermost layer and the lowermost layer were taken immediately after the preparation of the composition for transplantation and after 24-hour storage. The results presented in FIG. 8 show that adipocytes are uniformly present in the uppermost layer and the lowermost layer, while they precipitated down to the lowermost layer after 24-hour of storage.

The transplantation composition of the present invention comprises stably dispersed adipose stem cells, adipocytes and/or adipose tissues in a physiologically compatible physiological saline solution without showing a supernatant or precipitated adipose stem cells and/or adipocytes. Therefore, the transplantation composition of the present invention enables a precise control of the content of transplant cells, and may be injected into various transplantation regions in an exact amount, thereby markedly reducing side effects.

1. A composition for transplantation in a physiologically compatible buffer solution comprising:
(a) a transplant-cell selected from the group consisting of an adipose stem cell, an adipocyte and a mixture thereof; and
(b) a semi-solid substance derived from a living body or a biodegradable substance selected from the group consisting of hyaluronic acid, collagen, thrombin, elastin, chondroitin sulfate, albumin and a mixture thereof.

2. The composition for transplantation of claim 1, wherein the transplant-cell further comprises adipose tissues.

3. The composition for transplantation of claim 1, wherein the transplant-cell is a mixture of adipose stem cells, adipocytes and adipose tissues.

4. The composition for transplantation of claim 3, wherein the semi-solid substance derived from a living body or a biodegradable substance is contained in an amount of 0.0001-10 wt % relative to the total weight of the composition.

5. The composition for transplantation of claim 1, wherein the transplant-cell is an adipose stem cell; and the semi-solid substance derived from a living body or a biodegradable substance is contained in an amount of 0.001-20 wt % relative to the total weight of the composition.

6. The composition for transplantation of claim 1, wherein the transplant-cell is an adipocyte; and the semi-solid substance derived from a living body or a biodegradable substance is contained in an amount of 0.0001-10 wt % relative to the total weight of the composition.

7. The composition for transplantation according to claim 1, wherein the transplant-cell is an adipose stem cell, an adipocyte or an adipose tissue without damaged adipocytes.

8. The composition for transplantation according to claim 1, wherein the semi-solid substance derived from a living body or a biodegradable substance is a hyaluronic acid with a weight average molecular weight of 200,000-400,000.

9. The composition for transplantation according to claim 1, wherein the semi-solid substance derived from a living body or a biodegradable substance is collagen with a molecular weight of 100-200 kDa.
